(19) United States
(12) Patent Application Publication
(10) Pub. No.: US 2004/0202698 A1
(43) Pub. Date: Oct. 14, 2004

(54) Drug delivery systems comprising an encapsulated active ingredient

(75) Inventors: Niranjan Ramji, Mason, OH (US); Dana Paul Gruenbacher, Fairfield, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/405,420

(22) Filed: Apr. 2, 2003

(51) Int. Cl. A61K 9/70; A61K 9/68

(52) U.S. Cl. 424/443; 424/48

(57) ABSTRACT

The present invention is directed to drug delivery systems which comprise an active ingredient encapsulated within an edible film, wherein the edible film preferably provides for the buccal administration of the active into the bloodstream. The drug delivery systems of the present invention are especially effective in providing for the buccal administration of an encapsulated pharmaceutical active that is highly efficacious in providing sedation and/or sleep benefits.

### FIELD OF THE INVENTION

The present invention is directed to drug delivery systems which comprise an encapsulated active ingredient, wherein the active ingredient is preferably absorbed by oral mucosal membranes to result in buccal administration of the active into the bloodstream. In particular, the present invention is directed to drug delivery systems that comprise edible films which provide for the buccal administration of an encapsulated pharmaceutical active that is especially effective in providing sedation and/or sleep benefits.

### BACKGROUND OF THE INVENTION

There exist many ingestible pharmaceutical products which are suitable for treatment of symptoms related to the common cold, influenza, weight loss, sleep disorders, sinus, allergies, plaque, gingivitis, halitosis, and so forth. Other marketable ingestible pharmaceutical products include vitamin supplements, dietary supplements, diuretics, antiepileptics, hypnotics, and so forth. In addition to multiple ingestible pharmaceutical products on the market, there exist various ingestible cosmetic and oral care products including, breath freshners, breath-freshening gums, liquid-filled breath lozenges, mouth sprays, denture adhesives, teeth whitening products, plaque removal products, products for cavity prevention and treatment, tartar removal products, and so forth. These products comprise an active ingredient that is typically administered using drug delivery systems.

Drug delivery systems suitable for the delivery of actives are generally in the form of liquids, powders, capsules, films, drops, elixirs, sprays, syrups, chewing gums, and so forth. Common ingredients of these systems include water, and water-miscible solvents such as ethanol and polyethylene glycol. In essence, drug delivery systems are utilized to deliver actives to sites such as the oral cavity to provide a therapeutic benefit. For example, in the case of pharmaceutical actives, the pharmaceutical active can be delivered using a commonly employed technique known as buccal administration to result in the delivery of the active into the bloodstream.

The process of buccal administration is believed to involve the transmucosal delivery of actives such as pharmaceutical actives directly into the bloodstream without the active being first absorbed in the gastrointestinal tract and then delivered into the bloodstream. Buccally administered compositions are generally formulated to comprise a liquid content for absorption by mucosal membranes to result in the transmucosal delivery of the active. The liquid content, which typically comprise the active, can be administered in its liquid form, or the liquid can be included in a liquid elixir, a gelatin capsule, in a syrup formulation, in liquid sprays, and so forth, or the liquid can be admixed with polymeric film-forming agents for transmucosal delivery of the active.

Recent interest to consumers has been the administration of active ingredients, especially pharmaceutical actives, that are incorporated into polymeric film matrices for absorption of the active into the bloodstream via ingestion of the active into the gastrointestinal tract or by buccal administration. It has been shown that buccal administration can provide for rapid absorption of actives such as pharmaceutical actives into the bloodstream to achieve consumer acceptable therapeutic benefits. Therefore, attempts have been made to formulate drug delivery systems comprising an active ingredient and polymeric film wherein the active is incorporated within the film for buccal administration of the active to result in the active being delivered directly into the bloodstream.

One attempt of buccal administration of active ingredients using polymeric films is disclosed in U.S. Pat. No. 5,948,430. This disclosure describes compositions that comprise pharmaceutical or cosmetically active ingredients admixed with a water-soluble polymeric film-forming agent such as hydropypropyl methycellulose wherein the film composition is coated and then dried for administration in the oral cavity. The film compositions disclosed in U.S. Pat. No. 5,948,430 are described as monolayer films which are stated to rapidly soften and completely disintegrate in the oral cavity for application to the oral mucosa.

Another attempt of buccal administration of active ingredients using polymeric films is disclosed in U.S. Pat. No. 3,972,995, which describes edible films that comprise an active ingredient and layers composed of water-soluble or water-insoluble polymeric film-forming agents. The edible films disclosed in U.S. Pat. No. 3,972,995 are stated as being edible films which can adhere to moist body surfaces such as the interior surfaces of the mouth for an extended period.

Yet, another attempt of buccal administration of active ingredients using polymeric films is disclosed in WO 02/43657. WO 02/43657 discloses edible films that comprise active ingredients and film-forming agents other than a pullulan film-forming agent. The edible films described in WO 02/43657 are typically made by adding the active ingredients to a solution containing the film-forming agent, and then pouring this mixture onto a glass plate to form a thin film.

Still yet, another attempt at formulating an edible film that is suitable for oral consumption is disclosed in U.S. Pat. No. 6,419,903. The edible film disclosed in U.S. Pat. No. 6,419,903 is described as being suitable for the delivery of breath freshening agents, wherein the film is formed from a homogeneous mixture of the breath freshening agent, a hydroxyalkylmethyl cellulose film-forming polymer (e.g. hydroxypropylmethyl cellulose or HPMC), and a water disperspible starch.

Other disclosures of edible films comprising polymeric film materials include U.S. Pat. No. 4,623,394. U.S. Pat. No. 4,623,394 discloses molded articles including edible molded articles that exhibit controlled disintegration under hydrous conditions and that comprise a known polymeric pullulan film-forming agent in combination with a heteromannan. The molded articles disclosed in U.S. Pat. No. 4,623,394 can further comprise one or more substances such as a pharmaceutical active and tasting agents.

There also exist prior disclosures of capsules that contain polymeric film materials, wherein the capsules are suitable for use in the oral administration of an active ingredient to provide a therapeutic benefit. Such capsule based products include those described in U.S. Pat. No. 6,352,719; U.S. Pat. No. 6,238,696; U.S. Pat. No. 6,214,378; U.S. Pat. No. 6,413,463; U.S. Published Application 2001/0036473; WO 01/10443; WO 02/39980; WO 8504100; and the published article entitled “Advantages to HPMC Capsules: A New Generation's Hard Capsule” by Shunji Nagata in Drug Delivery Technology, March/April 2002, Vol. 2, No. 2.

It has been found that although a trend in the art is to deliver active ingredients using drug delivery systems such as those comprising polymeric film materials, the need still exists for a drug delivery system that comprises an edible film that provides for the improved delivery of active ingredients. As such, drug delivery systems that comprise active ingredients encapsulated with an edible film have been developed and found to be especially effective in the delivery of these active ingredients, especially those actives administered to treat symptoms related to sleep disorders. These drug delivery systems are particularly suitable for the buccal administration of active ingredients.

### SUMMARY OF THE INVENTION

The present invention is directed to drug delivery systems which comprise (a) an active ingredient, and (b) a water-soluble edible film, wherein the active is encapsulated within the edible film.

The present invention is also directed to a method of making drug delivery systems that are suitable for the administration of an active ingredient to the oral cavity, particularly for the buccal administration of active ingredients, wherein the method of making these drug delivery systems comprises the steps of (a) preparing a water-soluble edible film made from polymeric film-forming agents, and (b) encapsulating an active ingredient within the edible film.

It has been found that improved delivery of active ingredients such as those actives which provide sedation and/or sleep benefits can be achieved by drug delivery systems which comprise the active encapsulated within an edible film. Drug delivery systems that comprise actives encapsulated within an edible film have been shown to provide rapid disintegration of the film to result in the active being readily delivered and/or absorbed in the oral cavity, particularly absorbed by the mucosal membranes in the oral cavity for delivery of the active into the bloodstream.

### DETAILED DESCRIPTION OF THE INVENTION

The drug delivery systems of the present invention comprise an active ingredient encapsulated within an edible film. A preferred active ingredient includes one or more actives typically employed to treat symptoms related to sleep disorders, wherein the drug delivery systems are especially effective in providing for the buccal administration of these actives.

The term “active ingredient” as used herein refers to compounds or materials which are known or are otherwise effective in providing a therapeutic benefit. A “therapeutic benefit” is any measurable, noticeable, or perceived change in a symptom or condition that generates a reduction and/or feeling of relief from the symptom or condition. The “active ingredient” includes those known or otherwise effective pharmaceutical, cosmetic, and oral care compounds or materials that are suitable for oral consumption by mammals, including humans, pets, and domestic animals, to treat an unwanted symptom or condition.

The term “edible” as used herein refers to oral consumption by mammals. The “edible” materials defined herein include an individual edible ingredient, combinations of edible ingredients, and combinations of inedible and edible ingredients, provided that the edible and inedible ingredients are safe for mammalian consumption and do not provide any harmful adverse reactions.

The term “sleep disorders” as used herein refer to the onset, duration, and/or after effect of sleep or the lack of sleep. In other words, “sleep disorders” refer to, but is not limited to, the time to fall asleep, the lack of ability to fall asleep, amount of sleep, interrupted sleep, fragmented sleep, effective sleep, efficiency of sleep, insomnia, lack of restfulness, on and off sleep, and the tired, sluggish, or exhaustive feeling due to the lack of sleep. The term “sleep” is typically referred to as the state of rest that occurs periodically and that is characterized by relative physical nervous inactivity, lessened responsiveness, unconsciousness, and typical brain wave patterns as measured using electroencephalography.

The term “buccal administration” as used herein refers to application to the oral cavity. The oral cavity involves surfaces of, near, relating to, or lying in the mouth, including the mucosal membranes than line the mouth, throat, and tongue.

The terms “ambient temperature and “ambient conditions” are used interchangeably herein to refer to surrounding conditions at about one atmosphere of pressure, at about 50% relative humidity, at about 25° C.

The drug delivery systems of the present invention can comprise, consist of, or consist essentially of the elements and limitations of the invention described herein, as well as any of the additional or optional ingredients, components, or limitations described herein.

All documents cited herein, including publications, patent applications, and issued patents mentioned herein, are, in relevant part, incorporated herein by reference. Citation of any document is not an admission regarding any determination as to its availability as prior art to the present invention.

### Active

The drug delivery systems of the present invention comprise an active ingredient, preferably a pharmaceutical active ingredient, more preferably a pharmaceutical active ingredient that provides a therapeutic benefit in the treatment of symptoms related to sleep disorders. The active ingredient is present in the drug delivery systems as an encapsulated active, wherein suitable encapsulation materials are described hereinbelow.

The drug delivery systems of the present invention comprise the active ingredient as an individual active or as a combination of actives, provided that the total active content is capable of encapsulation using the encapsulation materials describe herein. For example, the drug delivery systems of the present invention can comprise one or more pharmaceutical actives, one or more cosmetic actives, one or more oral care actives, or combinations of one or more pharmaceutical actives, and/or one or more cosmetic actives, and/or one or more oral care actives.

The active ingredient is included in the drug delivery systems of the present invention as an encapsulated active, however, prior to encapsulation the active can be formulated in compositions which comprise other ingredients such as active solvents. In other words, the encapsulated active can be included in the drug delivery systems of the present invention as a solid encapsulated active, as an encapsulated active dissolved or dispersed in a semi-solid medium, or as an encapsulated active dissolved or dispersed in a liquid solution.

When the drug delivery systems of the present invention comprise a solid encapsulated active, the concentration of the active typically ranges from about 0.1 milligrams (mgs) to about 400 mgs, preferably from about 0.1 mgs to about 50 mgs, more preferably from about 5.0 mgs to about 25 mgs, by weight of the drug delivery system.

Preferably, the drug delivery systems of the present invention comprise the active ingredient as an encapsulated active that has been previously dispersed or dissolved in a semi-solid medium. Prior to encapsulation, the active ingredient is preferably dissolved or dispersed in hydrophilic, hydrophobic, or combination of these materials, to form a semi-solid medium. Suitable hydrophilic materials include, but are not limited to, those hydrophilic compounds which have a melting temperature of from about 25° C. to about 56° C., specific nonlimiting examples of which include polyethylene glycol (PEG) 600, PEG 800, PEG 1000, PEG 1450, and mixtures thereof. Suitable hydrophobic materials include glyceryl monooleate, triglycerol monooleate, and mixtures thereof. Typically, the active ingredient is included in the semi-solid medium at concentrations ranging from about 0.01% to about 50%, preferably from about 0.1% to about 20%, more preferably from about 0.5% to about 10%, even more preferably from about 1% to about 9%, by weight of the semi-solid medium. Other solubilization or dispersing agents can also be included in the semi-solid medium, nonlimiting examples of which include water, polysorbate 80, vegetable oil, mineral oil, peanut oil, soybean oil, and mixtures thereof.

The active ingredient suitable for use herein can also be dissolved or dispersed in a liquid solution prior to encapsulation of the solution. Liquids that are suitable for forming a solution with the active include solvents such as water-in-oil emulsion liquids, vegetable oil, mineral oil, lecithins including soy lecithin and lyso lecithin, and mixtures thereof. Typically, the active ingredient is included in the liquid solution at concentrations ranging from about 0.01% to about 50%, preferably from about 0.1% to about 20%, more preferably from about 0.5% to about 10%, even more preferably from about 1% to about 9%, by weight of the liquid solution.

The active ingredient suitable for use herein includes any known or otherwise effective compounds and materials commonly employed for use as a pharmaceutical, cosmetic, or oral care active to provide a therapeutic benefit. In general, pharmaceutical active ingredients are typically administered to treat symptoms resulting from discomforting and/or chronic illnesses such as symptoms of the common cold, influenza, sleep disorders, sinus, allergies, epilepsy, and so forth. Cosmetic active ingredients are generally administered to provide an aesthetically pleasing therapeutic benefit such as breath freshening and teeth whitening benefits. Oral care active ingredients are generally administered to treat symptoms related to plaque, gingivitis, halitosis, cavity prevention and treatment, cold sores, and so forth. As previously stated, the drug delivery systems of the present invention can comprise one or more pharmaceutical actives, one or more cosmetic actives, one or more oral care actives, or combinations of these active ingredients. The pharmaceutical active, cosmetic active, and oral care active ingredients are suitable for use in drug delivery systems that are orally consumed, however, these actives can also be incorporated into topical drug delivery systems or any other known or otherwise effective pharmaceutical, cosmetic, or oral care composition. Suitable pharmaceutical, cosmetic, and oral care active ingredients are described in detail hereinbelow.

Pharmaceutical Active

Nonlimiting examples of pharmaceutical actives suitable for use as an active ingredient herein include sedatives, hypnotics, antiepileptics, awakening agents, muscle relaxants, psychoneurotropic agents, neuromuscular blocking agents, antispasmodic agents, antiallergenics, cardiotonics, antiarrhythmics, diuretics, hypotensives, vasopressors, vasodilators, hemostats, thyroid hormones, sexual hormones, antidiabetics, antipychotics, antitumor agents, antibiotics, antiemetics, chemotherapeutics, steroids, immunosuppressants, narcotics, vitamin supplements, dietary supplements, antitussives, antihistamines, non-sedating antihistamines, decongestants, expectorants, mucolytics, analgesics, antipyretics, anti-inflammatory agents, and mixtures thereof. Preferred pharmaceutical actives suitable for use as an active ingredient herein include sedatives, hypnotics, vasodilators, antitussives, antihistamines, non-sedating antihistamines, decongestants, expectorants, mucolytics, analgesics, antipyretics, anti-inflammatory agents, and mixtures thereof. Sedatives and hypnotics are the most preferred pharmaceutical actives.

Specific nonlimiting examples of sedatives and hypnotics suitable for use as a preferred pharmaceutical active ingredient herein include those sedatives and/or hypnotics which can provide for a therapeutic benefit in the treatment of sleep disorders. Suitable specific sedatives and hypnotics include doxylamines including doxylamine succinate, melatonins, benzodiazepines including midazolam and triazolam, barbiturates, piperazines, clonidines, nitroglycerins, imidazopyridines, pyrazolopyrimidines, pharmaceutical salts thereof, and mixtures thereof. Doxylamines are most preferred. An example of a commercially available preferred doxylamine pharmaceutical active is doxylamine succinate commercially available from Ganes Chemicals Ltd. located in Pennsville, N.J., USA.

Specific nonlimiting examples of antibiotics suitable for use as a pharmaceutical active ingredient herein include augmentin, amoxicillin, tetracycline, doxycycline, minocycline, metronidazole, and mixtures thereof.

Specific nonlimiting examples of antitussives suitable for use as a pharmaceutical active ingredient herein include those antitussive compounds which are especially effective in treating symptoms of the common cold such as fits of coughing. Suitable specific antitussives include codeine, dextromethorphan, dextrorphan, hydrocodone, noscapine, oxycodone, pentoxyverine, and mixtures thereof. If the drug delivery systems of the present invention comprise an antitussive pharmaceutical active ingredient, dextromethorphan is the most preferred antitussive. As used herein, “dextromethorphan” means racemethorphan, (±)-3-Methoxy-17-methylmorphinan, dl-cis-1,3,4,9,10,10a-hexahydro-6-methoxy-11-methyl-2H-10,4a-iminoethanophenanthrene, and pharmaceutical salts thereof including dextromethorphan hydrobromide. Dextromethorphan and its pharmaceutically-acceptable salts are more fully described in U.S. Pat. No. 5,196,436, issued to Smith on Mar. 23, 1993, which description is incorporated by reference herein.

Specific nonlimiting examples of antihistamines suitable for use as a pharmaceutical active ingredient herein include acrivastine, azatadine, brompheniramine, brompheniramine maleate, chlorpheniramine, chlorpheniramine maleate, clemastine, cyproheptadine, dexbrompheniramine, dimenhydrinate, diphenhydramine, diphenhydramine hydrochloride, hydroxyzine, meclizine, pheninamine, phenyltoloxamine, promethazine, pyrilamine, pyrilamine maleate, tripelennamine, triprolidine, and mixtures thereof.

Specific nonlimiting examples of non-sedating antihistamines suitable for use as a pharmaceutical active ingredient herein include astemizole, cetirizine, ebastine, fexofenadine, loratidine, terfenadine, and mixtures thereof.

Specific nonlimiting examples of decongestants suitable for use as a pharmaceutical active ingredient herein include phenylpropanolamine, pseudoephedrine, pseudoephedrine hydrochloride, pseudoephedrine sulfate, ephedrine, phenylephrine, phenylephrine hydrochloride, oxymetazoline, and mixtures thereof

Specific nonlimiting examples of expectorants suitable for use as a pharmaceutical active ingredient herein include ammonium chloride, guafenesin, ipecac fluid extract, potassium iodide, and mixtures thereof.

Specific nonlimiting examples of mucolytics suitable for use as a pharmaceutical active ingredient herein include acetylcycsteine, ambroxol, bromhexine, and mixtures thereof.

Specific nonlimiting examples of analgesics, antipyretics, and anti-inflammatory agents suitable for use as a pharmaceutical active ingredient herein include acetaminophen, aspirin, sodium salicylate, salicylamide, diclofenac, diflunisal, etodolac, fenoprofen, flurbiprofen, ibuprofen, ketoprofen, ketorolac, nabumetone, naproxen, piroxicam, caffeine, ketorolac, indomethacin, meclofenamic acid, COX-2 inhibitors such as valdecoxib, celecoxib and rofecoxib, and mixtures thereof.

Cosmetic Active

Nonlimiting examples of cosmetic actives suitable for use as an active ingredient herein include breath freshening compounds commonly used for oral hygiene, actives used for dental and/or oral cleansing agents, teeth whitening agents, teeth stain bleaching agents, teeth stain removal agents, and mixtures thereof.

Specific nonlimiting examples of breath freshening compounds suitable for use as a cosmetic active ingredient herein include menthols, spearmints, spearmint oils, peppermints, peppermint oils, wintergreens, wintergreen oils, cinnamon derivatives, carboxamides, cyclic sulphones, sulphoxides, and mixtures thereof. Menthols and carboxamides are preferred breath freshening compounds.

Nonlimiting examples of commercially available menthols suitable for use as a preferred breath freshening, cosmetic active ingredient herein include 3-1-menthoxypropane-1,2-diol available as TK-10 from Takasago Perfumery Co., Ltd., Tokyo, Japan; menthone glycerol acetal available as MGA from Haarmann and Reimer; menthyl lactate available as Frescolat® from Haarmann and Reimer; and mixtures thereof.

Nonlimiting examples of commercially available carboxamides suitable for use as a preferred breath freshening, cosmetic active ingredient herein include the paramenthan carboxamides available under the WS-3 and WS-23 tradenames. WS-3 is commonly referred to as N-ethyl-p-menthan-3-carboxamide, and is available from Sterling Organics. WS-3 is more fully described in U.S. Pat. No. 4,136,163 issued to Watson et al. on Jan. 23, 1979, which description is incorporated herein by reference. WS-23 is commonly referred to as N,2,3-trimethyl-2-isopropylbutanamide. Mixtures of WS-3 and WS-23 are also suitable for use herein.

Specific nonlimiting examples of teeth whitening agents suitable for use as a cosmetic active ingredient herein include peroxides including hydrogen peroxide, urea peroxide, and calcium peroxide; perborates; percarbonates including sodium percarbonate; peroxyacids; persulfates including potassium, ammonium, sodium, and lithium persulfates; metal chlorites including calcium chlorite, barium chlorite, magnesium chlorite, lithium chlorite, sodium chlorite, and potassium chlorite; hypochlorite; chlorine dioxide; and mixtures thereof.

Oral Care Active

Nonlimiting examples of oral care actives suitable for use as an active ingredient herein include anticalculus agents, anticaries agents, antimicrobial agents, dentinal desensitizing agents, anesthetic agents, H-2 antagonists, nutrients, and mixtures thereof. Examples of the oral conditions these actives address include, but are not limited to, appearance and structural changes to teeth, plaque removal, tartar removal, cavity prevention and treatment, inflamed and/or bleeding gums, mucosal wounds, lesions, ulcers, aphthous ulcers, cold sores, tooth abscesses, and so forth.

Specific nonlimiting examples of anticalculus agents suitable for use as an oral care active ingredient herein include polyphosphates and salts thereof including tripolyphosphates, tetrapolyphosphates, potassium hydrogen phosphate, sodium hydrogen phosphate, and sodium hexametaphosphate; pyrophosphates and salts thereof including trisodium pyrophosphates, disodium dihydrogen pyrophosphate, dipotassium pyrophosphate, tetrasodium pyrophosphate, and tertapotassium pyrophosphate; polyamino propane sulfonic acid (AMPS) and salts thereof; polyolefin sulfonates and salts thereof; polyvinyl phosphates and salts thereof; polyolefin phosphates and salts thereof; diphosphonates and salts thereof including azocycloalkane-2,2-diphosphonic acids and salts thereof, ions of azocycloalkane-2,2-diphosphonic acids and salts thereof (such as those which the alkane moiety has five, six or seven carbon atoms, in which the nitrogen atom is unsubstituted or carries a lower alkyl substitutent, e.g. methyl), azacyclohexane-2,2-diphosphonic acid, azacyclopentane-2,2-diphosphonic acid, N-methyl-azacyclopentane-2,3-diphosphonic acid, EHDP (ethanehydroxy-1,1,-diphosphonic acid), AHP (azacycloheptane-2,2-diphosphonic acid, a.k.a. 1-azocycloheptylidene-2,2-diphosphonic acid), ethane-1-amino-1,1-diphosphonate, and dichloromethane-diphosphonate; phosphonoalkane carboxylic acid and salts thereof including phosphonopropane tricarboxylic acid (PPTA) and phosphonobutane-1,2,4-tricarboxylic acid (PBTA); polyphosphonates and salts thereof; polyvinyl phosphonates and salts thereof including polyvinylphosphonic acid; polyolefin phosphonates and salts thereof including those wherein the olefin group contains 2 or more carbon atoms; polypeptides including polyaspartic and polyglutamic acids; and mixtures thereof.

Specific nonlimiting examples of anticaries agents suitable for use as an oral care active ingredient herein include xylitol, fluoride ion source, and mixtures thereof. It is believed that the fluoride ion source provides free fluoride ions during the use of the drug delivery systems of the present invention, wherein the fluoride ion source is selected from the group consisting of sodium fluoride, stannous fluoride, indium fluoride, organic fluorides such as amine fluorides, sodium monofluorophosphate, and mixtures thereof. The fluoride ion source can provide for fluoride ions in the range of from about 50 parts per million (ppm) to about 10,000 ppm, more preferably from about 100 ppm to about 300 ppm, when the drug delivery systems of the present invention contact dental surfaces. The fluoride ion source is more fully described in U.S. Pat. No. 2,946,725 issued to Norris et al. on Jul. 26, 1960; and U.S. Pat. No. 3,678,154 issued to Widder et al. on Jul. 18, 1972; which descriptions are incorporated herein by reference.

Specific nonlimiting examples of antimicrobial agents suitable for use as an oral care active ingredient herein include alexidine; chlorhexidine including chlorhexidine hydrochloride; hexetidine; sanguinarine; hexylresorcin; benzalkonium chloride; dequalinium chloride; cetypyridinium chloride (CPC); tetradecylpyridinium chloride (TPC); ethacridine; salicylanilide; domiphen bromide; triclosan; sodium chlorite; N-tetradecyl-4-ethylpyridinium chloride (TDEPC); piperidino derivatives including octenidine, delmopinol, and octapinol; methyl salicylate; antimicrobial metals and salts thereof for example those providing zinc ions, stannous ions, and copper ions; bisbiguanides; phenolics; anti-fungals such as those for the treatment of candida albicans; analogs thereof; salts thereof; and mixtures thereof.

Specific nonlimiting examples of dentinal desensitizing agents suitable for use as an oral care active ingredient herein include strontium chloride; potassium nitrate; natural herbs such as gall nut, Asarum, Cubebin, Galanga, scutellaria, Liangmianzhen, Baizhi; and mixtures thereof.

Specific nonlimiting examples of H-2 antagonists suitable for use as an oral care active ingredient herein include cimetidine, etintidine, ranitidine, ICIA-5165, tiotidine, ORF-17578, lupitidine, donetidine, famotidine, roxatidine, pifatidine, lamtidine, BL-6548, BMY-25271, zaltidine, nizatidine, mifentidine, BMY-25368 (SKF-94482), BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408, burimamide, metiamide, and mixtures thereof.

Specific nonlimiting examples of nutrients suitable for use as an oral care active ingredient herein include minerals including calcium, phosphorus, fluoride, zinc, manganese, and potassium; oral care vitamins including Vitamin C, Vitamin D, thiamine, riboflavin, calcium pantothenate, niacin, folic acid, nicotinamide, pyridoxine, cyanocobalamin, para-aminobenzoic acid, and bioflavonoids; oral nutritional supplements including amino acids, lipotropics, and fish oil; enteral nutritional supplements including protein products, glucose polymers, corn oil, safflower oil, and medium chain triglycerides; and mixtures thereof.

The present invention is directed to drug delivery systems that comprise an encapsulated active ingredient. However, it is contemplated that the drug delivery systems of the present invention are suitable for incorporation into a composition or product that contains unencapsulated active ingredients. Suitable unencapsulated active ingredients include those actives that are present in the drug delivery systems of the present invention as an encapsulated active ingredient.

### Edible Film

The drug delivery systems of the present invention comprise an edible film that provides for the encapsulation of the active ingredient described hereinabove. The active ingredient is encapsulated into the edible film such that upon administration of the edible film into the oral cavity the edible film rapidly disintegrates and provides for the release of the active ingredient in the oral cavity, preferably for absorption of the active by the oral mucosal membranes. The administration of an encapsulated active ingredient results in the improved delivery of the actives for absorption by the oral mucosal membranes.

The edible film suitable for use herein preferably comprise polymeric film-forming agents that are water-soluble, and that are capable or rapid disintegration in an aqueous environment such as the oral cavity. The edible film can comprise the water-soluble, polymeric film-forming agents in combination with water-insoluble or water dispersible materials, provided that the resultant edible film can disintegrate in an aqueous environment to result in dissolution of the edible film. The terms “edible film” and “edible films” are used interchangeably herein to include the singular and plural forms of these terms.

The disintegration of the edible film can be measured using any known or otherwise effective technique to measure the break-up or decomposition of polymeric film-forming agents. A suitable technique involves adding the edible film into a beaker containing about 50 milliliters (mls) of saliva, artificial saliva, or a combination thereof, and then stirring the edible film and saliva mixture at about 400 revolutions per minute (rpm) while maintaining the temperature of the mixture at about 37° C. until the edible film breaks-up or dissolves. Another suitable technique to measure the break-up or decomposition of polymeric film-forming agents include placing an edible film onto the tongue of a human volunteer who has abstained from food or drink one hour prior to consumption of the edible film, and then recording the time of dissolution of the edible film as perceived by the human volunteer. The edible film suitable for use herein disintegrates in an aqueous environment such as the oral cavity in about 20 seconds to about 120 seconds, preferably about 30 seconds to about 60 seconds, to result in the dissolution of the edible film for release of the encapsulated active ingredient into the oral cavity.

The edible film suitable for use herein can comprise one or more layers of polymeric film-forming agents, provided that the resultant edible film is capable of undergoing rapid disintegration. Typically, a suitable edible film has a film thickness of from about 0.01 millimeters (mm) to about 0.1 mm, preferably from about 0.04 mm to about 0.08 mm. If the edible film comprises multiple layers, the film thickness of each layer generally ranges from about 0.005 mm to about 0.06 mm, however, the thickness of the resultant edible film ranges from about 0.01 mm to about 0.1 mm. Unlike capsules made from polymeric film-forming agents, the edible film suitable for use herein can be described as thin-film encapsulation materials that rapidly disintegrates in an aqueous environment, preferably the edible film is characterized as a thin film that rapidly disintegrates and releases an encapsulated active ingredient upon the buccal administration of the edible film into the oral cavity.

The edible film suitable for use herein can be administered in various polymeric product forms including edible pouches, edible sachets, edible strips, edible rods, edible blister packs, edible stickpacks, and so forth. The product forms can be manufactured in shapes of round, oval, square, tubular, elliptical, conical, cylindrical, hemispherical, and the like, provided that these forms and shapes are edible films having the requisite film thickness defined herein.

As previously stated, the edible film suitable for use herein comprise water-soluble polymeric film-forming agents. Nonlimiting examples of suitable water-soluble polymeric film-forming agents include polymeric cellulose derivatives, natural polymers, polymeric gums, pullulans, poloxamers, polyvinyl pyrrolidones (PVPs), dextran polymers, carboxypolymethylenes, carboxyvinyl polymers, copolymers of polymethyl vinyl ether and maleic anhydride, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, and mixtures thereof. Water-soluble, polymeric cellulose derivatives are the preferred polymers for forming the edible film defined herein.

Specific nonlimiting examples of water-soluble, polymeric cellulose derivatives suitable for use as a preferred polymeric film-forming agent herein include hydroxypropylmethyl cellulose (HPMC), hydroxyethyl cellulose (HEC), ethylhydroxyethyl cellulose (EHEC), hydroxypropyl cellulose (HPC), methyl cellulose, carboxymethyl cellulose (CMC), salts of carboxymethyl cellulose including the sodium salt of carboxymethyl cellulose, and mixtures thereof. Hydroxypropylmethyl cellulose is the most preferred polymeric film-forming agent.

Preferably, the edible film defined herein comprises a hydroxypropylmethyl cellulose that has a viscosity of from about 3.0 milliPascal-seconds (mPa•s) to about 50 mPa•s, preferably from about 3.0 mPa•s to about 6.0 mPa•s, as measured using a 2% solution of hydroxypropylmethyl cellulose. It has been found that edible films that comprise a hydroxypropylmethyl cellulose film-forming agent having a viscosity within the above defined range can rapidly disintegrate in an aqueous environment to provide for the improved delivery of an active ingredient, particularly the improved delivery of an active ingredient into the oral cavity. The viscosity of the hydroxypropylmethyl cellulose can be determined by any known or otherwise effective technique for determining viscosity. A suitable technique involves the use of a Ubbelohde tube viscometer that can determine viscosity of aqueous polymeric solutions at 20° C.

Specific examples of commercially available preferred hydroxypropylmethyl cellulose film-forming agents include the hydroxypropylmethyl celluoses that are commercially available from the Dow Chemical Company (Midland, Mich., USA) under the Methocel E5 Premium LV and Methocel E3 Premium LV tradenames. Methocel E5 and Methocel E3 Premium LVs are USP grade, low viscosity hydroxypropylmethyl celluloses that have 29.1% methoxyl groups and 9% hydroxypropyl group substitution. Methocel E5 Premium LV is commercially available as a white or off-white free-flowing dry powder that typically has a viscosity of about 5.1 mPa•s as measured using a Ubbelohde tube viscometer to determine the viscosity of a 2% by weight aqueous solution of Methocel E5 Premium LV at 20° C. Methocel E3 Premium LV is also commercially available as a white or off-white free flowing dry powder that typically has a viscosity of about 3.0 mPa•s measuring a 2% solution of the polymeric film-forming agent using the Ubbelohde tube viscometer technique.

Specific nonlimiting examples of water-soluble, natural polymers and polymeric gums suitable for use as polymeric film-forming agents herein include arabic gums, tragacanth gums, agar polymers, xanthan gums, copolymers of alginic acid and sodium alginate, chitosan polymers, pectins, carageenans, modified starches, and mixtures thereof.

Specific nonlimiting examples of water-soluble, poloxomers suitable for use as a polymeric film-forming agent herein include those poloxamers commercially available under the Lutrol F-127 and Lutrol F-68 tradenames, and mixtures thereof.

Specific nonlimiting examples of water-soluble, copolymers of polymethyl vinyl ether and maleic anhydride suitable for use as a polymeric film-forming agent herein include those copolymers commercially available under the Gantrez tradename including Gantrez S and Gantrez MS type copolymers, and mixtures thereof.

Specific nonlimiting examples of water-soluble, homopolymers of acrylic acid crosslinked with an allyl ether of pentaerythritol or an allyl ether of sucrose suitable for use as polymeric film-forming agents herein include those homopolymers commercially available from the B. F. Goodrich Company under the tradename “Carbopol”. Specific Carbopols include Carbopol 934, 940, 941, 956, 980, and mixtures thereof. Carbopol 980 is preferred among the Carbopol film-forming agents.

Specific nonlimiting examples of water-soluble, homopolymers of acrylic acid crosslinked with divinyl glycol suitable for use as a film-forming agent herein include those homopolymers commercially available from the B. F. Goodrich Company as polycarbophils under the tradename “Noveon.”

The water-soluble, polymeric film-forming agents suitable for use herein are more fully described in the following publications: Journal Pharmacy Pharmacology 53, (2001 Edition); the International Journal of Pharmaceutics (1988, 1996 and 1998 Editions); and the Journal Controlled Release 62, (1999 Edition); which descriptions are incorporated herein by reference.

The water-soluble, polymeric film-forming agents can be used alone or in combination to form the edible film herein, however, the edible film typically comprise the polymeric film-forming agents at total polymer concentrations ranging from about 5% to about 70%, preferably from about 15% to about 70%, more preferably from about 20% to about 65%, by weight of the edible film.

Likewise, when the edible film comprises multiple layers, each edible film layer can comprise concentrations of the water-soluble, polymeric film-forming agents as defined hereinabove wherein each edible film layer can comprise the same or different combinations of polymeric film-forming agents. For example, (1) the edible film herein can be a monolayer edible film comprising one polymeric film-forming agent or a combination of polymeric film-forming agents, (2) the edible film herein can be a bilayer edible film comprising one layer of one polymeric film-forming agent or a combination of polymeric film-forming agents, and another layer of one polymeric film-forming agent or a combination of polymeric film-forming agents, or comprising a combination thereof, or (3) the edible film herein can comprise three or more layers comprising various possible combinations of polymeric film-forming agents wherein the possible combinations include layers comprising one polymeric film-forming agent, a combination of polymeric film-forming agents, or combinations of these layers. Preferably, the edible film herein is a bilayer edible film comprising an inner first layer of one or more polymeric film-forming agents and an outer second layer of one or more polymeric film-forming agents.

### Optional Components

The drug delivery systems of the present invention may further comprise one or more optional components known or otherwise effective for use in pharmaceutical, cosmetic, and oral care compositions, provided that the optional components are physically and chemically compatible with the active ingredient and edible film components described hereinabove, or do not otherwise unduly impair product stability, aesthetics, or performance. The optional components can be included with the encapsulated active ingredient, as components of the edible film, or both the encapsulated active ingredient and edible film can comprise optional components. Optional components suitable for use herein include materials such as flavoring agents, sweeteners, chelating agents, preservatives, sensates, processing aids including sodium alginate, buffers including sodium glycinate, and pH adjusting aids including triethanolamine, L-lysine, L-arginine, and sodium hydroxide. The optional components can be included at concentrations ranging from about 0.001% to about 15%, preferably from about 0.1% to about 10%, by weight of the drug delivery system.

The drug delivery systems of the present invention can optionally comprise homeopathic ingredients. A detailed, but not necessarily a complete list, of such homeopathic ingredients is found in The Homeopathic Pharmacopoeia of the United States, 1999 ed., published by The Pharmacopoeia Convention of the American Institute of Homeopathy, ©1982, Vol. 1-4, which descriptions are incorporated herein by reference. Specific nonlimiting examples of known, homeopathic, or otherwise effective, optional components suitable for use herein are described in more detail hereinbelow.

A specific nonlimiting example of an optional component suitable for use herein include optional flavoring agents. The optional flavoring agents can provide for drug delivery systems which have a non-bitter, or other aesthetically pleasing tastes. Suitable optional flavoring agents include, but are not limited to, anise, eucalyptus, oil of clove, lemon, lime, honey, honey lemon, red fruit, grapefruit, orange, grape, cherry, cherry cola, berry, and mixtures thereof. If present, the optional flavoring agents are generally included at concentrations ranging from about 0.001% to about 1.0% by weight of the drug delivery system.

Another specific nonlimiting example of an optional component suitable for use herein include optional sweeteners selected from the group consisting of aspartame; saccharin and its salts including calcium saccharin and sodium saccharin; Sucralose™ (sold by the McNeil Specialty Products Co., New Brunswick, N.J.); Prosweet™ (sold by the Virginia Dare Extract Co., New York, N.Y.); Magnasweet™ (sold by MAFCO Worldwide Corp., Licorice Division, Camden, N.J.); ammonium glycyrrhizinate and its salts; Talin™ (Thaumatin) and its diluted products such as Talin GA90 (sold by the Talin Food Company, Birkenhead, England); Acesulfame K; and mixtures thereof. If present, the optional sweeteners are generally included at concentrations ranging from about 0.1% to about 2.0% by weight of the drug delivery system.

Yet, another specific nonlimiting example of an optional component suitable for use herein include chelating agents which are suitable for use in drug delivery systems that comprise an active ingredient that provides for antiviral benefits. It is believed that the optional chelating agent can provide for enhanced antiviral activity, wherein suitable optional chelating agents include those that chelate transition metal ions such as iron, copper, zinc and other such metals. Not to be bound by theory, it is reasonable to postulate that metal ions, specifically metal cations, play a major role in the formation of oxidizing species. Oxidizing reactions and free radical formation can contribute to cellular damage in inflammatory diseases. The optional chelating agents useful herein are known to dampen oxidation reactions. The optional chelating agents are stable and effective in non-aqueous and aqueous mediums. Nonlimiting examples of suitable optional chelating agents include phytic acid, disodium and calcium salts of ethylene diamine tetraacetic acid (EDTA), tetrasodium EDTA, sodium hexametaphosphate (SHMP), di(hydroxyethyl)glycine, 8-hydroxyquinoline, and mixtures thereof. If the drug delivery systems of the present invention comprise one or more optional chelating agents, the chelating agents are included at concentrations ranging from about 0.05% to 0.5%, preferably from about 0.05% to about 0.3%, more preferably from about 0.05% to about 0.15%, by weight of the drug delivery system.

Other specific nonlimiting examples of optional components suitable for use herein include optional preservatives. Preservatives can optionally be included to prevent any form of microbial contamination. Such optional preservatives include benzalkonium chloride, chlorhexidine gluconate, phenyl ethyl alcohol, phenoxyethanol, benzyl alcohol, sorbic acid, thimerosal, phenylmercuric acetate, and mixtures thereof.

### Method of Manufacture

The drug delivery systems of the present invention may be prepared by any known or otherwise effective technique suitable for providing an active ingredient encapsulated within an edible film. However, the drug delivery systems of the present invention are preferably prepared such that an encapsulated active ingredient is readily released upon administration of a drug delivery system into the oral cavity for absorption of the active by the oral mucosal membranes.

In general, the drug delivery systems of the present invention are prepared by constructing an edible film using known film-forming techniques including injection molding, extrusion molding, blow molding, compression molding, cast filming, spray filming, and dip filming methods. The resultant edible film is then filled with an active ingredient. Next, the edible film containing the active ingredient is sealed using any known or commonly employed heat sealing process to form a drug delivery system of the present invention.

A particular process of preparing drug delivery systems of the present invention comprises admixing materials to construct an edible film, wherein the admixture typically comprises one or more polymeric film-forming agents, one or more water-miscible solvents such as propylene glycol and polyethylene glycol, and optional ingredients such as glycerine, a sweetener, a flavoring agent, and a processing aid such as sodium alginate. The admixture is then constructed into an edible film using a cast filming technique which involves spreading the admixture onto a glass plate, and allowing the admixture to dry under ambient conditions to form a film. For edible films comprising multiple layers, an admixture as described hereinabove is spread onto a glass plate and allowed to dry followed by the spreading of an identical or different admixture layer. The dried edible film is removed from the glass plate, and then formed into a desired shape using known techniques such as thermoforming. Next, the formed edible film is filled with an active ingredient, and sealed using known heat-sealing processes such as sealing using ultrasound, lasers, or microwave heat-sealing equipments. Dependent on the desired shape and form, the resultant active encapsulated edible film can be in the form of a cup, pouch, sachet, blister pack, stickpack, and so forth. An exemplary cup-shaped edible film is constructed such that another admixture layer of edible film is positioned atop of the edible film cup to create a cover for the cup, wherein the cover and cup are heat-sealed into a drug delivery system of the present invention. Alternatively, the edible films can be constructed in the shape or form of pouches, tubes, sachets, stickpacks, blister packs, and so forth, wherein each shape or form comprise an encapsulated active ingredient to result in drug delivery systems of the present invention.

The edible films can encapsulate a solid active ingredient, an active ingredient dispersed or dissolved in a semi-solid medium, or an active ingredient dispersed or dissolved in a liquid solution. For active ingredients dispersed or dissolved in a semi-solid medium, the dispersion or dissolution process typically involves adding the active ingredient to a mixture of semi-solid materials such as a mixture of PEG 600 and PEG 1450, and heating with stirring the resultant mixture of active and semi-solid materials to a temperature of from about 30° C. to about 55° C. until the active is dispersed or dissolved throughout the mixture. For active ingredients dispersed or dissolved in a liquid solution, the dispersion or dissolution process typically involves adding the active ingredient to an liquid solution of mineral oil or vegetable oil and stirring the resultant solution at 25° C. until the active is dispersed or dissolved in the oil.

The drug delivery systems of the present invention are suitable for the administration of an encapsulated active ingredient into the oral cavity, preferably for the buccal administration of an encapsulated active ingredient. The type and form of active ingredient typically determines the amount or concentration of active ingredient encapsulated within the edible films described herein. Semi-solid mediums and liquid solutions of active ingredients are the preferred forms of active ingredient for encapsulation within the edible films described herein. Therefore, typically from about 0.1 mls to about 1.0 mls of semi-solid mediums or liquid solutions are encapsulated within an edible film to result in from about 1.0 mgs to 50 mgs of active ingredient administered into the oral cavity.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Semi-solid mediums and liquid solutions of active ingredient are exemplified hereinbelow in Tables I and II. The semi-solid mediums and liquid solutions are prepared by mixing one or more active ingredients with semi-solid and/or liquid materials to result in an active ingredient that is suitable for encapsulation by an edible film exemplified herein below in Table III. The semi-solid mediums or liquid solutions of active ingredient are capable of encapsulation within an edible film for improved delivery of the active ingredient into the oral cavity to result in absorption of the active ingredient by the oral mucosal membranes.

TABLE I
Solutions of Active Ingredient

| | Sample 1 | Sample 2 | Sample 3 | Sample 4 |
|---|---|---|---|---|
| Component | (Wt. %) | (Wt. %) | (Wt. %) | (Wt. %) |
| Doxylamine Succinate¹ | 8.05 | 8.06 | 3.18 | 3.33 |
| Carboxamide WS-3² | 0.04 | 0.04 | — | — |
| PEG 600³ | 60.21 | 40.1 | 45.58 | — |
| PEG 1450⁴ | 20.07 | 40.1 | 45.58 | 92.3 |
| L-lysine⁵ | 8.0 | 8.0 | 3.18 | 1.66 |
| Polysorbate 80⁶ | 2.10 | 2.10 | 2.48 | — |
| Water | — | — | — | 2.71 |
| Artificail Cherry | 1.03 | 1.10 | — | — |
| Flavor #27239⁷ | | | | |
| Sucralose⁸ | 0.50 | 0.50 | — | — |
| Total: | 100 | 100 | 100 | 100 |

Wt. %—weight percent
¹Doxylamine succinate active ingredient available from Ganes Chemicals Ltd., Pennsville, NJ
²Carboxamide WS-3 active ingredient available from Mellenium Speciality Chemicals, Jacksonville, FL
³PEG 600 is a polyethylene glycol available from Dow Chemical/Plaquemine LA
⁴PEG 1450 is a polyethylene glycol available from Union Carbide Corporation, Danbury, CT
⁵L-lysine is available from Sigma Aldrich Co, St. Louis, MO
⁶Polysorbate 80 available from Mallinckrodt Baker Inc, Paris, KY
⁷Cherry flavor available from Bush Boake Allen, Norwood, NJ
⁸Sucralose available from McNeil Speciality products

TABLE II
Solutions of Active Ingredient

| | Sample 5 | Sample 6 | Sample 7 | Sample 8 |
|---|---|---|---|---|
| Component | (Wt. %) | (Wt. %) | (Wt. %) | (Wt. %) |
| Doxylamine Succinate¹ | 4.75 | 6.95 | 4.76 | 3.12 |
| Carboxamide WS-3² | 0.20 | 0.04 | — | — |
| Dextromethorphan | — | 16.68 | — | — |
| hydrobromide⁹ | | | | |
| PEG 400¹⁰ | — | 51.81 | — | — |
| PEG 1450⁴ | — | 17.27 | — | — |
| L-lysine⁵ | — | 6.82 | — | — |
| Glyceryl monoleate | 28.52 | — | — | 93.76 |
| (Dimodan)¹¹ | | | | |
| Triglycerol monooleate¹² | 66.53 | — | — | — |
| Mineral oil¹³ | — | — | 66.66 | — |
| Precept 8160¹⁴ | — | — | 28.58 | — |
| Sodium glycinate¹⁵ | — | — | — | 3.12 |
| Sucralose⁸ | — | 0.43 | — | — |
| Total: | 100 | 100 | 100 | 100 |

Wt. %—weight percent
⁹Dextromethorphan hydrobromide available from Hoffman Laroche, Inc., Nutley, NJ
¹⁰PEG 400 is a polyethylene glycol available from the Union Carbide Corporation
¹¹Glyceryl monooleate available from Danisco cultor, USA Inc.
¹²Triglycerol monooleate available from Abitec Corp.
¹³Mineral oil available from Witco Chemicals, PA
¹⁴Precept 8160 is an enzyme modified soy lecithin available from Central soya, Fort Wayne, IN
¹⁵Sodium glycinate available from Sigma Aldrich Co., St. Louis, MO

TABLE III
Edible Film

| | Sample 9 | Sample 10 | Sample 11 | Sample 12 |
|---|---|---|---|---|
| Component | (Wt. %) | (Wt. %) | (Wt. %) | (Wt. %) |
| HPMC E3 Premium | 67.31 | 67.64 | 74.65 | 82.60 |
| LV¹⁶ | | | | |
| Xanthan gum¹⁷ | 5.04 | 5.63 | 6.22 | 6.19 |
| Carboxymethyl | — | — | 8.29 | — |
| cellulose sodium¹⁸ | | | | |
| Alginic acid sodium | — | 7.51 | — | — |
| copolymer¹⁹ | | | | |
| Propylene glycol²⁰ | 18.51 | — | — | — |
| PEG 600³ | — | 7.51 | 8.29 | 8.26 |
| Glycerine²¹ | 8.41 | 5.66 | 1.65 | 2.06 |
| L-lysine⁵ | — | 3.75 | — | — |
| Carboxamide WS-3² | 0.20 | 0.20 | 0.20 | 0.20 |
| Sucralose⁸ | 0.53 | 0.60 | 0.70 | 0.69 |
| Titanium dioxide | — | 0.75 | — | — |
| (Rutile)²² | | | | |
| FD&C Blue #1²³ | — | 0.75 | — | — |
| Total: | 100 | 100 | 100 | 100 |

Wt. %—weight percent
¹⁶Hydroxypropylmethyl cellulose available from Dow Chemical Company, Midland, MI
¹⁷Xanthan gum available from CP Kelco Inc, Okmulgee, OK
¹⁸Carboxymethyl cellulose available from Sigma Aldrich Co, St. Louis, MO
¹⁹Alginic acid sodium available from Sigma Aldrich Co, St. Louis, MO
²⁰Propylene glycol available from Dow, Plaquemine, LA
²¹Glycerine available from Procter & Gamble Co, Cincinnati, OH
²²Titanium dioxide available from Kronos, Varennes, Quebec, Canada
²³FD&C Blue available from B. F. Goodrich, Hilton-Davis, Cincinnati, OH

### Examples I-III

Drug delivery systems of the present invention are exemplified hereinbelow in Table IV. The drug delivery systems are prepared by encapsulating an active ingredient exemplified in Table I or Table II within an edible film exemplified in Table III. Final product forms of these drug delivery systems include pouches, stickpacks, blister packs, and so forth, wherein the drug delivery systems are administered into the oral cavity. These drug delivery systems are especially effective for administration into the oral cavity to treat symptoms associated with sleep disorders.

TABLE IV

| Component | Drug Delivery System |
|---|---|
| Example I | Active Ingredient of Sample 1 encapsulated |
| | within a bilayer Edible Film |
| | made from Sample 9 and Sample 10. |
| Example II | Active Ingredient of Sample 8 encapsulated |
| | within a bilayer Edible Film |
| | made from Sample 9 and Sample 10. |
| Example III | Active Ingredient of Sample 1 encapsulated |
| | within a bilayer Edible Film |
| | made from Sample 10 and Sample 11. |

While particular embodiments of the present invention have been described, it will be obvious to those skilled in the art that various changes and modifications of the present invention can be made without departing from the spirit and scope of the invention. It is intended to cover, in the appended claims, all such modifications that are within the scope of this invention.

What is claimed is:

1. A drug delivery system comprising:
(a) an active ingredient; and
(b) a water-soluble edible film;
wherein the active is encapsulated within the edible film.

2. The drug delivery system of claim 1 wherein the active ingredient is a selected from the group consisting of a pharmaceutical active, a cosmetic active, an oral care active, and mixtures thereof.

3. The drug delivery system of claim 2 wherein the pharmaceutical active is selected from the group consisting of sedatives, hypnotics, antiepileptics, awakening agents, muscle relaxants, psychoneurotropic agents, neuromuscular blocking agents, antispasmodic agents, antiallergenics, cardiotonics, antiarrhythmics, diuretics, hypotensives, vasopressors, vasodilators, hemostats, thyroid hormones, sexual hormones, antidiabetics, antipychotics, antitumor agents, antibiotics, antiemetics, chemotherapeutics, steroids, immunosuppressants, narcotics, vitamin supplements, dietary supplements, antitussives, antihistamines, non-sedating antihistamines, decongestants, expectorants, mucolytics, analgesics, antipyretics, anti-inflammatory agents, and mixtures thereof.

4. The drug delivery system of claim 3 wherein the pharmaceutical active is a hypnotic pharmaceutical active selected from doxylamine, a pharmaceutically salt thereof, and mixtures thereof.

5. The drug delivery system of claim 4 wherein the pharmaceutical active is doxylamine succinate.

6. The drug delivery system of claim 2 wherein the cosmetic active is selected from the group consisting of breath freshening compounds, dental cleansing agents, oral cleansing agents, teeth whitening agents, teeth stain bleaching agents, teeth stain removal agents, and mixtures thereof.

7. The drug delivery system of claim 6 wherein the breath freshening compounds are selected from the group consisting of menthols, spearmints, spearmint oils, peppermints, peppermint oils, wintergreens, wintergreen oils, cinnamon derivatives, carboxamides, cyclic sulphones, sulphoxides, and mixtures thereof.

8. The drug delivery system of claim 2 wherein the oral care active is selected from the group consisting of anticalculus agents, anticaries agents, antimicrobial agents, dentinal desensitizing agents, anesthetic agents, H-2 antagonists, nutrients, and mixtures thereof.

9. The drug delivery system of claim 1 wherein the active ingredient is dissolved or dispersed in a semi-solid medium.

10. The drug delivery system of claim 1 wherein the active ingredient is dissolved or dispersed in a liquid solution.

11. The drug delivery system of claim 1 wherein the water-soluble edible film comprises a water-soluble polymeric film-forming agent selected from the group consisting of polymeric cellulose derivatives, natural polymers, polymeric gums, pullulans, poloxamers, polyvinyl pyrrolidones (PVPs), dextran polymers, carboxypolymethylenes, carboxyvinyl polymers, copolymers of polymethyl vinyl ether and maleic anhydride, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, and mixtures thereof.

12. The drug delivery system of claim 11 wherein the polymeric cellulose derivatives are selected from the group consisting of hydroxypropylmethyl cellulose, hydroxyethyl cellulose, ethylhydroxyethyl cellulose, hydroxypropyl cellulose, methyl cellulose, carboxymethyl cellulose, salts of carboxymethyl cellulose, and mixtures thereof.

13. The drug delivery system of claim 12 wherein the polymeric cellulose derivative is hydroxypropylmethyl cellulose having a viscosity of from about 3.0 mPa•s to about 50 m mPa•s.

14. The drug delivery system of claim 11 wherein the water-soluble edible film comprises one or more layers of water-soluble polymeric film-forming agent.

15. The drug delivery system of claim 14 wherein the water-soluble edible film has a film thickness of from about 0.01 mm to about 0.1 mm.

16. A method of making a drug delivery system, the method comprising the steps of:
(a) constructing a water-soluble edible film; and
(b) encapsulating an active ingredient within the edible film.

17. The method of claim 16 wherein the water-soluble edible film is constructed from a water-soluble polymeric film-forming agent.

18. The method of claim 17 wherein the water-soluble polymeric film-forming agent is selected from the group consisting of polymeric cellulose derivatives, natural polymers, polymeric gums, pullulans, poloxamers, polyvinyl pyrrolidones (PVPs), dextran polymers, carboxypolymethylenes, carboxyvinyl polymers, copolymers of polymethyl vinyl ether and maleic anhydride, homopolymers of acrylic acid crosslinked with an allyl ether of pentaerythritol, homopolymers of acrylic acid crosslinked with an allyl ether of sucrose, homopolymers of acrylic acid crosslinked with divinyl glycol, and mixtures thereof.

19. The method of claim 16 wherein the active ingredient is selected from the group consisting of a pharmaceutical active, a cosmetic active, an oral care active, and mixtures thereof.

20. The method of claim 19 wherein the active ingredient is dissolved or dispersed in a semi-solid medium.

21. The method of claim 16 wherein the drug delivery system is administered into the oral cavity.
